(19) United States
(12) Patent Application Publication
(10) Pub. No.: US 2017/0112742 A1
(43) Pub. Date: Apr. 27, 2017

(54) TOPICAL COMPOSITION FOR TREATING ALOPECIA

(71) Applicant: Jacquelyn Turner, Tampa, FL (US)

(72) Inventor: Jacquelyn Turner, Tampa, FL (US)

(21) Appl. No.: 14/921,003

(22) Filed: Oct. 23, 2015

(51) Int. Cl. A61K 8/49; A61K 8/34; A45D 40/04; A61K 8/98; A61M 35/00; A61Q 7/00; A61K 8/92

(52) U.S. Cl. CPC A61K 8/4953; A61Q 7/00; A61K 8/34; A61K 8/345; A61K 8/922; A61K 8/985; A61M 35/003; A45D 40/04; A61K 2800/87; A61K 2800/43; A61K 2800/592

(57) ABSTRACT

A topical composition for treating alopecia is provided. The topical composition includes a combination of a hair growth solution and a moisturizer. The topical composition has a semi-solid to solid form.

### BACKGROUND OF THE INVENTION

The composition described herein relates to the field of hair treatment, more particularly, to topical alopecia treatment.

Male pattern baldness (MPB) affects roughly 40 million men in the United States. Approximately one in four men will have noticeable hair loss by age 30, and this figure increases to two in three men by age 60. In rare cases, MPB can begin as early as age 12.

A majority of both men and women agree that signs of thinning, shedding, or balding hair make them feel more self conscious than being overweight, having skin or complexion problems, or having crooked teeth.

There is a wide range of products that claim to make thin hair or alopecia appear thicker and healthier for cosmetic purposes. In spite of this wide availability, there is no “magic pill” to cure baldness, and the currently available products all have serious drawbacks and imperfect performance.

Currently available products generally fall into three categories: hair growth stimulants, hair fiber products, and hair color products. Hair growth stimulant products do not color hair or disguise hair loss. Hair fiber products do not color hair and do not stimulate hair growth. Hair color products do not stimulate hair growth.

One commonly used hair growth stimulant is minoxidil. One major drawback of minoxidil is that it must be applied to the scalp using an ethanol or propylene glycol topical solution. This can cause dryness and may worsen the appearance of the user's hair.

There remains a need in the art for a topical solution that colors hair, adds bulk to hair, and stimulates hair growth while not drying out the scalp.

### SUMMARY OF THE INVENTION

In one aspect of the present invention, a topical composition for treating alopecia, comprising a hair growth solution and a moisturizer, wherein the topical composition comprises a semi-solid to solid form.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown with a cap covering the topical composition;

FIG. 2 is an exploded view of an embodiment of the present invention;

FIG. 3 is a perspective view of an embodiment of the present invention demonstrating dial operation;

FIG. 4 is a front view of an embodiment of the present invention in use;

FIG. 5 is a front view of an embodiment of the present invention in use; and

FIG. 6 is a schematic view of an embodiment of the development of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a composition that includes a hair re-growth solution combined with a butter, a color pigment and micro hair fibers. Minoxidil reinvigorates shrunken hair follicles, which increases their size and helps re-grow thicker looking hair over time. It helps reverse the progression of hereditary hair loss. The color pigment stains the scalp to help give the immediate appearance of thickness and to cover grey hair. The micro hair fibers cling to the existing hair and scalp to also give the immediate appearance of fuller, thicker looking hair. The essential butters nourish dry hair and scalp.

The present invention utilizes one or more “fats” as the base that are typically used for making of cosmetic moisturizers, salve, or oil. However, “fats” may also be understood to include other non-traditional butter-like materials, or binders i.e., nontoxic solids at room temperature that melt at body temperature. The pigments may be based on any cosmetically acceptable pigments. Most frequently, the pigments are based on metallic oxides, such as yellow, black, brown, or red iron oxides, white titanium dioxide, chromium oxide or a botanical pigment or mixture of any combination thereof. Other useful materials for incorporation include, but are not limited to, ceremides, lipids, minoxidil, finestride, saw palmetto and herbal/botanical dye, tint, rinse, instant color or butters.

The present invention includes a topical composition for treating alopecia. The topical composition includes a combination of a hair growth solution and a moisturizer. The topical composition has a semi-solid to solid form.

The hair growth solution may include a combination of at least one of minoxidil and propecia with at least one of ethanol and propylene glycol with water. The ethanol assists in dissolving the minixoidil into a liquid form. The ethanol and water influence the penetration into the skin of the minoxidil which stimulates hair growth. As the concentration of minoxidil is increased, so is the amount of ethyl alcohol (ethynol). The ethanol helps act as a carrier and thus influences the absorption of minoxidil into the skin.

The moisturizer may include any fat based carrier. The fat based carrier may be a butter. The butter may include, but is not limited to, shea butter, vitamin E butter, kukui butter, neem butter, aloe butter and a combination thereof. Further, the present invention may include a sow wax to add an additional adherence to the composition. The butters help absorb moisture from the air and hold it in the skin and counter the drying effect of the ethanol.

The present invention may further include a pigment. The pigment includes a hair color. The pigment adds color to skin where the hair is missing or thinning to camouflage the gaps. The pigment coats the current hair which makes it appear thicker and fuller. The hair color may include, but is not limited, to one of the following: a brown color; a black color; a red color; and a blonde color.

In certain embodiments, the present invention may further include a plurality of hair fibers. The hair fibers cling to the existing hair and scalp and make the hair appear thicker and fuller. The plurality of hair fibers may be substantially evenly distributed within the composition.

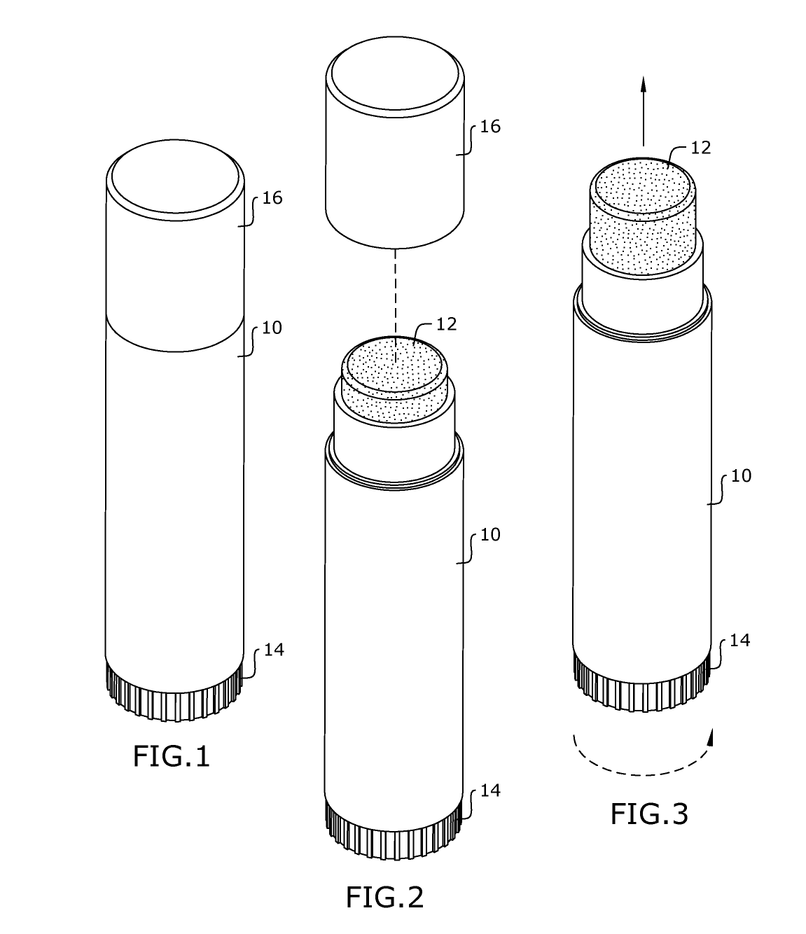

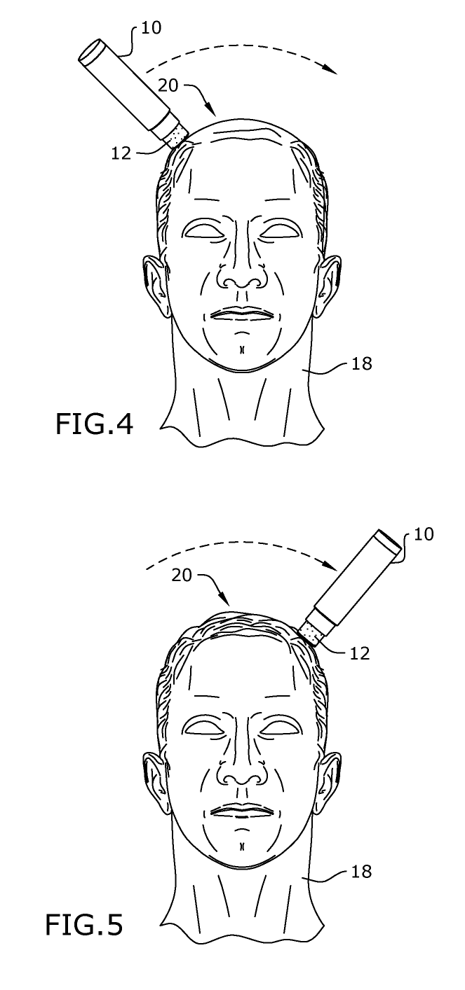

Referring to FIG. 1 through 5, the topical composition may be in the form of a stick 12. The stick 12 may be disposed within a stick casing 10. A turn dial 14 may be used to urge the stick 12 outside of and back within the casing 10. A cap 16 is releasably attachable to the top of the casing 10, thereby covering and preserving the stick 12 within. The use the above embodiments, a user 18 may remove the cap 16 and urge the stick 12 outside of the casing 10. The user 18 may then rub the stick over the hair loss region 20. Since the stick includes a pigment and a plurality of hair fibers, the present invention provides noticeable improvement instantly. Further, the hair re-growth solution with provide either prevention of additional hair loss and/or hair re-growth.

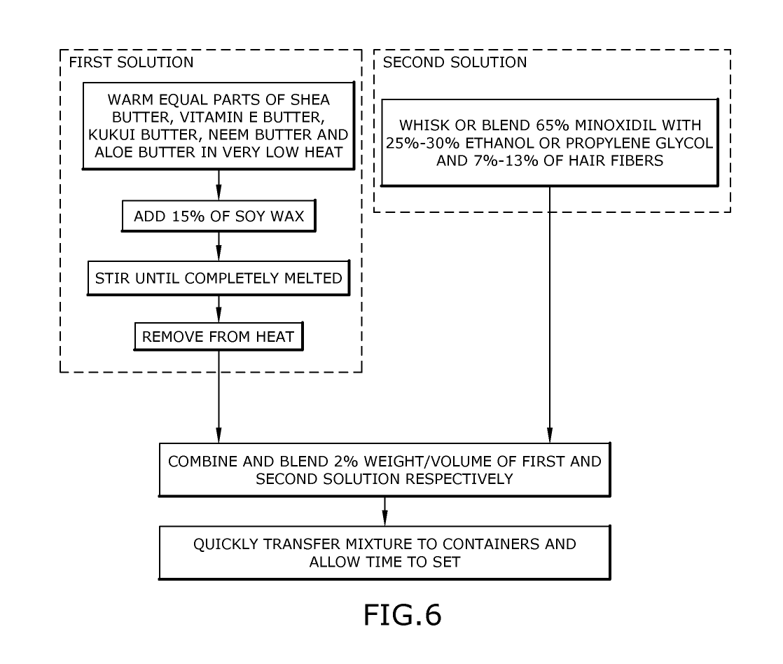

A method of making the present invention may include the following. Referring to FIG. 6, a first solution is formed. The first solution may include warming equal parts of the butters at a low heat. Add 15% of soy wax. The combination of butters and soy wax may make up 60% or more of this final formulation. Stir until completely melted. Remove from heat. A second solution is formed by whisking or blending together about 65% of the hair re-growth product with about 25-30% of the ethanol or propylene glycol and with about 7-13% of hair fibers. The first solution and the second solution are combined to form the composition such that the second solution is about 2% to about 5% of the total combined composition. Once blended, quickly transfer the mixture into containers and allow time to set. The present invention may be applied straight from the formed product. Simply rub the product across the affected area. The present invention may also be smudged to approximate the appearance of a fuller thicker hair immediately.

The present invention may form one of: a grease or oil that stimulates hair growth and adds color; a hair bond that stimulates hair growth and adds color; a hair mask that stimulates hair growth and adds color; a moisturizer that stimulates hair growth and adds color pigment; a shampoo and/or conditioner and stimulates hair growth and adds color; and a semi-permanent or permanent hair dye, rinse or tint product.

It should be understood, of course, that the foregoing relates to exemplary embodiments of the invention and that modifications may be made without departing from the spirit and scope of the invention as set forth in the following claims.

What is claimed is:

1. A topical composition for treating alopecia, comprising a hair growth solution and a moisturizer, wherein the topical composition comprises a semi-solid to solid form.

2. The topical composition of claim 1, wherein the hair growth solution comprises at least one of minoxidil and finastride combined with at least one of ethanol and propylene glycol.

3. The topical composition of claim 1, wherein the moisturizer is a fat based carrier.

4. The topical composition of claim 3, wherein the fat based carrier is a butter.

5. The topical composition of claim 4, wherein the butter is selected from a group consisting of shea butter, vitamin E butter, kukui butter, neem butter, aloe butter and a combination thereof.

6. The topical composition of claim 1, further comprising soy wax.

7. The topical composition of claim 1, further comprising a pigment comprising a hair color.

8. The topical composition of claim 7, wherein the hair color comprises one of a brown color, a black color, a red color and a blonde color.

9. The topical composition of claim 1, further comprising a plurality of hair fibers.

10. The topical composition of claim 9, wherein the plurality of hair fibers are substantially evenly distributed within the composition.

11. The topical composition of claim 1, wherein the semi-solid to solid form comprises a stick.

12. The topical composition of claim 11, further comprising a stick casing, wherein the stick is disposed within the casing.

13. The topical composition of claim 12, further comprising a turn dial attached to the casing, and operable to urge the stick out of the casing.

14. The topical composition of claim 12, further comprising a cap releasably attached to the casing and covering the stick.
